(19) United States
(12) Patent Application Publication
(10) Pub. No.: US 2021/0318287 A1
(43) Pub. Date: Oct. 14, 2021

(54) LOCALIZED SURFACE PLASMON RESONANCE SENSOR SYSTEMS AND METHODS

(71) Applicant: The Regents of the University of Michigan, Ann Arbor, MI (US)

(72) Inventors: Steven Kaye, Ann Arbor, MI (US); Walker McHugh, Ann Arbor, MI (US); Zhen Luo, Ann Arbor, MI (US); Andrew Fleszar, Ann Arbor, MI (US); Skyler Rietberg, Ann Arbor, MI (US)

(21) Appl. No.: 17/315,773

(22) Filed: May 10, 2021

Related U.S. Application Data
(63) Continuation-in-part of application No. 15/551,164, filed on Aug. 15, 2017.
(60) Provisional application No. 62/116,741, filed on Feb. 16, 2015.
(60) Provisional application No. 62/245,066, filed on Oct. 22, 2015.
(60) Provisional application No. 63/023,693, filed on May 12, 2020.

(51) Int. Cl. G01N 33/487; G01N 21/59; G01N 33/569; G01N 21/552

(52) U.S. Cl. CPC G01N 33/48707; G01N 21/59; G01N 2021/5903; G01N 21/553; G01N 33/56983

(57) ABSTRACT

The invention(s) cover a sensor and method of fabrication, the sensor including: a substrate; and a distribution of nanoparticles patterned onto the substrate as a set of regions. In variations, the sensor 100 can further include one or more channels in fluid communication with the distribution of nanoparticles. In variations, different nanoparticle regions can be optionally functionalized with different probe molecules in order to provide additional functionality with respect to the assay(s) being performed using the sensor 100. Additionally or alternatively, in variations, unoccupied regions of the substrate 110 and/or nanoparticle surfaces can optionally include passivated surfaces to prevent non-specific binding, without significantly shifting the LSPR wavelength, in order to significantly improve signal-to-noise ratio (SNR) provided by the sensor. The sensor can be used for performance of multiplexed assays (e.g., for infectious disease panels) with processing of different types of sample material.

### CROSS-REFERENCE TO RELATED APPLICATIONS

This application is a continuation-in-part U.S. patent application Ser. No. 15/551,164 filed 16 Feb. 2016, which claims priority to U.S. Provisional Patent Application No. 62/116,741 filed 16 Feb. 2015, and U.S. Provisional Patent Application No. 62/245,066 filed 22 Oct. 2015, which are each incorporated herein in its entirety by this reference.

This application also claims priority to U.S. Provisional Application No. 63/023,693, filed 12 May 2020, which is hereby incorporated in its entirety by this reference.

### TECHNICAL FIELD

This invention relates generally to the diagnostics field, and more specifically to new and useful localized surface plasmon resonance sensor systems and methods.

### BACKGROUND

Surface plasmons are the oscillations of electrons at a metal-dielectric interface. When this oscillation is confined to a nanoparticle of a size less than the wavelength of light corresponding to the resonant energy of the oscillation, it is known as localized surface plasmon resonance (LSPR), and the optical absorption at this resonant energy is both greatly enhanced and extremely sensitive to the dielectric field surrounding the nanoparticle, making it an excellent sensor material.

To form a solid-state LSPR sensor, nanoparticles are deposited on a substrate. A probe molecule (e.g., antibody) selective for the target analyte is then attached to the nanoparticles. When a sensor is exposed to the target analyte, that analyte binds to the probe, changing the dielectric field around the nanoparticle and therefore the nanoparticle's optical spectrum. This change in optical spectrum can be measured using a spectrometer or similar instrument. As such, solid-state LSPR sensors can provide rapid detection of target material for various assays (e.g., immunoassays, other binding assays, etc.) involving various sample formats.

Current sensors based on LSPR have significant drawbacks, some of which include: limitation in only containing a single antibody per sensor substrate; poor stability due to either aggregation of the nanoparticles or poor adhesion of nanoparticles to a substrate; subject to low signal due to: a) insufficient probe molecules (e.g. antibodies) attached to the nanoparticle, and/or b) too many probe molecules around the nanoparticles, such that the analyte is kept too far from the nanoparticle to impact the resonant energy; poor selectivity due to a) non-specific binding of the analyte to the nanoparticle surface, and/or b) non-specific binding of the probe to the sensor substrate; and/or other deficiencies.

Thus, there is a need in the sample processing field to create improved localized surface plasmon resonance sensor systems and methods.

### BRIEF DESCRIPTION OF THE FIGURES

FIG. 1 depicts a schematic of an embodiment of a localized surface plasmon resonance (LSPR) sensor.

FIG. 2 depicts a schematic of a portion of an embodiment of an LSPR sensor, including a passivated surface and an adhesion layer.

FIGS. 3A through 3E depict variations of nanoparticle/region types of an embodiment of an LSPR sensor.

FIG. 4 depicts an embodiment of a portion of an embodiment of an LSPR sensor, including an optional nanoparticle coating.

FIG. 5A depicts a flowchart of an embodiment of a method for manufacturing an LSPR sensor.

FIG. 5B depicts a flow diagram of a portion of an embodiment of a method for manufacturing an LSPR sensor.

FIG. 6 depicts a specific example of a portion of an LSPR sensor produced according to the embodiment shown in FIGS. 5A and 5B.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Benefits

The invention(s) can confer several benefits over conventional systems and methods.

For instance, embodiments of the invention(s) include sensors for rapid and multiplexed assays for various applications, where the sensors implement localized surface plasmon resonance (LSPR) structures in a compact format. Examples of the invention(s) include sensors for rapid, saliva-based assays for diseases and other health conditions, enabled by such LSPR technology. In particular, examples of the sensors can enable performance of saliva-based assays that can provide real-time results in less than 10 minutes in a low-cost disposable format. Such sensors can be configured for multiplexed assays for respiratory pathogen panels, with or without multiple antibodies per pathogen, in a compact format.

Examples of the invention(s) can include systems and methods for immunoassays for detection of target material associated with infection diseases, such as the detection of pathogen-specific antigens (e.g., SARS-CoV-2 spike protein, SARS-CoV-2 nucleoprotein) and for detection of host antibodies which arise as a results of the bodies natural immune response in response to infection (e.g., anti-SARS-CoV-2 spike protein IgG). Variations of the invention(s) can include systems and methods for immunoassays for detection of various target antibodies (e.g., IgG antibodies, IgM antibodies, IgA antibodies, IgD antibodies, IgE antibodies). Variations of the invention(s) can include systems and methods for immunoassays for detection of various target specific antigens (e.g., SARS-CoV-2 spike protein, SARS-CoV-2 nucleoprotein, respiratory syncytial virus glycoprotein, influenza A nucleoprotein).

Variations of the inventions(s) can include systems and methods for other assays associated with detection of other target material/probes.

Variation(s) of the invention(s) can accommodate other sample types from suitable biological and/or non-biological sources.

In embodiments, sensor systems covered by the invention(s) include: an optically transparent substrate; patterned regions of nanoparticles (e.g., monodisperse, high-density nanoparticles) with high density and without aggregation; one or more regions optionally functionalized with different probe molecules; and unoccupied substrate and nanoparticle surfaces optionally passivated to prevent non-specific binding without significantly shifting the LSPR wavelength. Such sensor systems can thus enable performance of multiplexed assays in a manner that significantly increases signal-to-noise ratio (SNR) in a reliable manner.

Additionally or alternatively, the system and/or method can confer any other suitable benefit.

### 2. Definitions

The terms “reagent”, “process material”, and “assay reagent” as used herein are used in the broadest sense and refers to any material useful, necessary, or sufficient for performing the assays of the present disclosure. Examples include, but are not limited to, antibodies, controls, buffers, calibration standards and the like.

The term “sample” in the present specification and claims is used in its broadest sense. It is meant to include both biological samples, environmental samples, and/or other suitable samples. A sample can further include material of synthetic origin.

Biological samples may be plant-associated, bacterium-associated, virus-associated, and/or animal-associated, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc.

Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present disclosure.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

“Antigen binding molecule” refers to a molecule that binds a specific antigen. Examples include, but are not limited to, proteins, nucleic acids, aptamers, synthetic molecules, etc. As used, the terms “probe” and “affinity molecule” can each cover an antigen binding molecule.

“Antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, single-chain variable fragments (scFv), and Fab expression libraries.

“Specific binding” or “specifically binding” when used in reference to the interaction of an antibody and an antigen means that the interaction is dependent upon the presence of a particular structure (e.g., the antigenic determinant or epitope) on the antigen; in other words the antibody is recognizing and binding to a specific structure rather than to antigens in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, “microfluidic” refers to, for example, a device for transport or storage of small volumes (e.g., of liquids such as assay reagents). In some embodiments, individual channels or chamber of microfluidic devices comprise a volume of 10 nL to 5000 μL, although other sizes can be implemented.

The terms “test compound” and “candidate compound” refer to any chemical entity, pharmaceutical, drug, and the like that is a candidate for use to treat or prevent a disease, illness, sickness, or disorder of bodily function. Test compounds comprise both known and potential therapeutic compounds. A test compound can be determined to be therapeutic by screening using the screening methods of the present disclosure.

### 3. Sensor

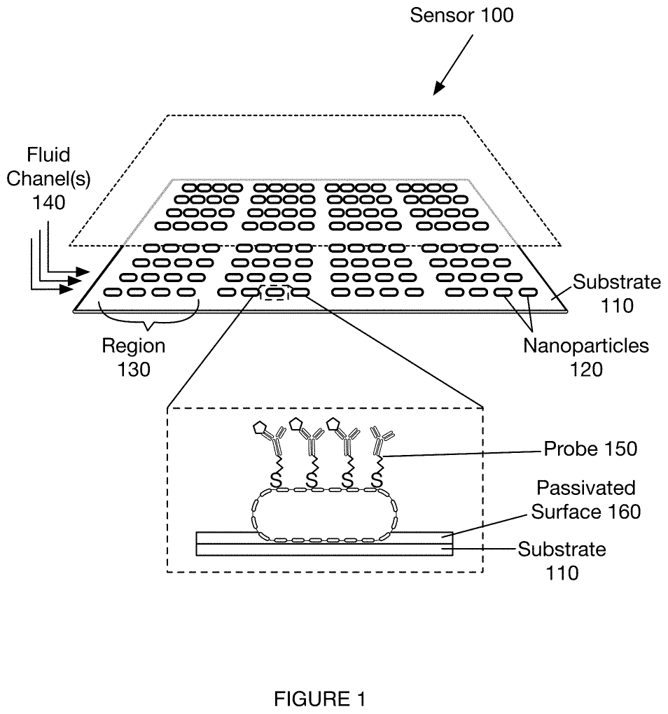

As shown in FIG. 1, an embodiment of a sensor 100 includes: a substrate 110; and a distribution of nanoparticles 120 patterned onto the substrate as a set of regions 130. In variations, the sensor 100 can further include one or more channels 140 in fluid communication with the distribution of nanoparticles 120. In variations, different nanoparticle regions can be optionally functionalized with different probe molecules 150 in order to provide additional functionality with respect to the assay(s) being performed using the sensor 100. Additionally or alternatively, in variations, unoccupied regions of the substrate 110 and/or nanoparticle surfaces can optionally include passivated surfaces 160 to prevent non-specific binding, without significantly shifting the LSPR wavelength, in order to significantly improve signal-to-noise ratio (SNR) provided by the sensor 100.

The sensor 100 includes functionalized nanoparticle structures for LSPR, where binding of target material to the functionalized nanoparticle structures results in changes in local indices of diffraction, which can be used to perform various assays. The sensor 100 thus provides a label-free mechanism for direct real-time binding detection. The sensor 100 also provides a solid-phase approach for multiplexed detection, with elimination of cross-talk/interference between detection regions for improved SNR. Embodiments of the sensor 100 are compact and can be used for point of care diagnostics, and can provide detection with small sample volumes and short assay times.

In variations, the sensor 100 functions to provide structures for performing immunoassays and/or other types of assays involving detection of target material from a sample. In examples, the sensor 100 can be configured to process biological samples (e.g., saliva samples, blood samples, urine samples, serum samples, other biological material) for detection of antibodies associated with respiratory diseases, such as antibodies against SARS-CoV-2.

Other variations of the sensor 100 can be used for applications including one or more of: therapeutic drug monitoring, infectious organism detection, diagnostics, detection of cancer biomarkers, detection of nutritional biomarkers, detection of genetic markers, detection of gene expression markers, detection of microRNA markers, hormone monitoring, fertility monitoring, serology testing for detection of various target antibodies (e.g., IgG antibodies, IgM antibodies, IgA antibodies, IgD antibodies, IgE antibodies), detection of cardiovascular health biomarkers, detection of gastrointestinal health biomarkers, detection of sexual health markers, detection of autoantibodies, detection of renal health biomarkers, and/or detection of other material associated with other conditions.

In embodiments, the sensor 100 functions to provide nanoparticle regions arranged for performance of multiplexed assays, with one or more of: control regions, replicate regions, epitope regions, regions configured for multiple targets, regions configured with components having different affinities for the same target, and/or other suitable regions.

The sensor 100 can be fabricated according to methods described in Section 4 below.

### 3.1 Sensor—Substrate

As shown in FIG. 1, the sensor 100 includes a substrate 110, which functions to provide one or more surfaces onto which the distribution of nanoparticles 120 is patterned (as described below). The substrate no also functions to facilitate detection of optical signals by having suitable optical characteristics for transmission of light signals generated from the sensor no to an optical signal sensing apparatus 111. The substrate no is preferably configured to transmit optical signals derived from scattering of light from functionalized nanoparticles, rather than from an illumination source used to excite the functionalized particles.

In embodiments, the substrate no is optically transparent. In one embodiment, the substrate no is composed of a material that provides greater than 90% transmission of light, and in variations, the substrate no is composed of a material that provides greater than 95% or greater than 99% transmission of light (e.g., greater than 99.5%, preferably greater than 99.9%). However, the substrate no can alternatively be composed of a material that provides other suitable light transmission characteristics.

Light transmission characteristics of the substrate no can be tuned/structured for transmission defined by wavelength ranges in which the distribution of nanoparticles 120 absorb light. In variations, light transmission characteristics can be tuned/structured for wavelength ranges in which the distribution of nanoparticles 120 absorb light, where the wavelength ranges are expanded (e.g., unidirectional, symmetrically bi-directional, asymmetrically bi-directional, etc.) in order to allow for dust/noise/artifact detection and mitigation. In examples, the wavelength ranges can be expanded by at least 100 nm, however, in other examples, the wavelength ranges can be expanded by another suitable value.

The substrate 110 can further be configured with a refractive index greater than 1 (e.g., greater than 1.3), and in a specific example, the substrate 110 is configured with a refractive index greater than 1.5. The refractive index can be specified to allow light to be transmitted through the substrate 110 by way of total internal reflectance; however, the substrate 110 can alternatively be configured with another suitable refractive index. Furthermore, in still alternative embodiments, different portions of the substrate can be configured with different refractive indices (e.g., to control and provide differential signal transmission characteristics from different regions of nanoparticles described below, to serve another suitable functions, etc.).

In one embodiment, the substrate 110 is composed of glass/silica (e.g., a borosilicate glass), which offers desired optical properties for optical detection/imaging, as well as providing a suitable level of surface modification capacity for surface functionalization. Alternative materials used for the substrate 110 can include, but are not limited to one or more of: plastic/polymer materials (e.g., acrylic, cyclic olefin polymer, polycarbonate, poly(methyl methacrylate) (PMMA), cyclo olefin polymer (COP), polystyrene, polypropylene, polyethylene terephthalate glycol-modified (PEGT), etc.); ternary compositions (e.g., indium tin oxide); and/or other suitable materials.

### 3.2 Sensor—Patterned Regions of Nanoparticles

As shown in FIG. 1, the sensor 100 further includes a distribution of nanoparticles 120 patterned onto the substrate 110 as a set of regions 130, where the nanoparticles 120 are functionalized with one or more affinity compounds (e.g., with multiple affinity compounds in the array of nanoparticles, with each nanoparticle region only including one affinity compound) for binding to target/non-target material components of a sample. As such, the distribution of nanoparticles 120 functions to enable performance of multiplexed and/or non-multiplexed binding assays, where binding changes light scattering characteristics of the functionalized particles in a manner that is detectable upon excitation by illumination by incident light.

In embodiments, the nanoparticles 120 can be composed of metals that support surface plasmons. In variations, the nanoparticles 120 can be composed of metallic materials, such as one or more of: gold, silver, copper, titanium, chromium, ruthenium, rhodium, palladium, osmium, iridium, platinum, and/or other suitable metallic materials (e.g., noble metals, metal core-shell structures, plasmonic metamaterials, etc.). Additionally or alternatively, the nanoparticles 120 can be composed of metal/semiconductor composites (e.g., chitosan-metal nanoparticles, graphene-metal nanoparticles, etc.). The material(s) of the nanoparticles 120 are preferably non-reactive with the samples (e.g., biological samples) being processed; however, the materials of the nanoparticles 120 can be otherwise configured.

In some embodiments, the nanoparticles 120 have the form of nanorods; however, in alternative embodiments, the nanoparticles 120 can have other geometric configurations (e.g., nanospheres, nanostars, nanodiamonds, nanopyramids, nanobipyramids, nanorings, metal core-shell structures, etc.).

In relation to patterning of the distribution of nanoparticles 120, patterning of nanoparticles 120 onto the substrate 110 can be achieved in multiple ways.

In a first variation, nanoparticles can be deposited onto the substrate in the desired pattern (e.g., by printing of a solution of nanoparticles onto the substrate no, by spray coating of nanoparticles onto the substrate no, etc.).

In a second variation, a thin (e.g., nanometer-thick) film/layer can be etched or ablated into the desired pattern onto the substrate.

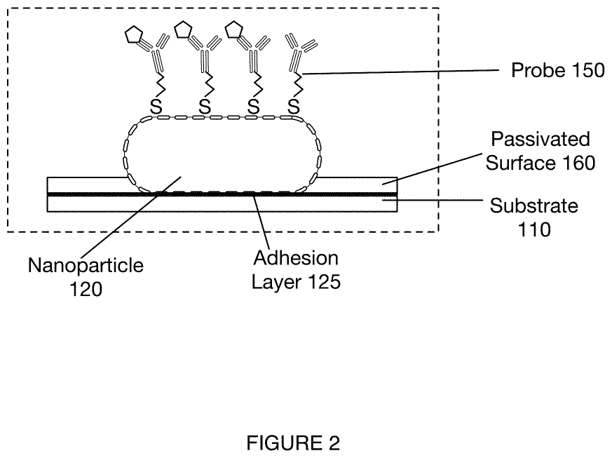

In a third variation, shown in FIG. 2, the sensor no can include an adhesion layer 125 having a high affinity for the nanoparticles and patterned onto the substrate no in a desired pattern. In this variation, the distribution of nanoparticles is coupled to the adhesion layer 125 in the desired pattern, thereby distributing nanoparticles across the substrate no in functionalized regions for various applications (described in further detail below). In order to have suitable affinity for the nanoparticles, the adhesion layer 125 can contain a functional group (e.g., amine, thiol, disulfide-modified, hydroxyl, carboxyl methyl, etc.); however, affinity for the nanoparticles can be achieved in another manner.

In more detail with respect to this variation, the substrate no can be pattern-coated with the adhesion layer having a high affinity for the nanoparticles, followed by a deposition process in which nanoparticles only adhere to the adhesion layer. In an example of this variation, photolithography can be used to apply a patterned resist onto the substrate 110, followed by deposition of an aminosilane adhesion layer in regions exposed by the patterned resist. Removal of the patterned resist then leaves a patterned aminosilane coating as the adhesion layer having high affinity for the nanoparticles. Immersion of the substrate 110 in a solution of gold nanoparticles then results in gold nanoparticles only adhering to the aminosilane adhesion layer.

However, patterning can be achieved using other suitable methods, embodiments, variations, and examples of which are further described in Section 4 below.

Preferably, the nanoparticles are patterned on the substrate no with as high a density as possible, in order to increase optical signal aspects without significant aggregation, which increases noise. In particular, the nanoparticles are preferably configurated with a density that produces desired optical signal aspects while preventing electromagnetic coupling between nanoparticles or regions of nanoparticles. In particular, with respect to density and aggregation, the distribution of nanoparticles 120 is preferably configured as monodisperse particles (e.g., uniformly distributed particles, particles having substantially uniform size and/or configuration, etc.), with a critical distance between particles to prevent electromagnetic coupling; however, in alternative variations, the distribution of nanoparticles 120 may not be configured as monodisperse particles.

In variations, the density of nanoparticles can range from 1 particles/μm² through 100 particles/μm², and in a specific example, the distribution of nanoparticles 120 has a density of 16 particles/μm². However, the distribution of nanoparticles 120 can alternatively have another suitable density.

In embodiments, the nanoparticles can be configured with a level of aggregation, where less than 10% of nanoparticles are positioned within 250 nm of another nanoparticle. In one variation, the nanoparticles can be configured with a level of aggregation, where less than 5% of nanoparticles are positioned within 250 nm of another nanoparticle. In still another variation, the nanoparticles can be configured with a level of aggregation, where less than 1% of nanoparticles are positioned within 250 nm of another nanoparticle; however, the nanoparticles can additionally or alternatively be configured with another suitable level of aggregation.

In patterning regions of nanoparticles, the regions are preferably separated by sufficient distance that they can be resolved by the optical detection/imaging system. In one variation, the center-to-center distance between regions is greater than ix the characteristic diameter of a respective region. In another variation, the center-to-center distance between regions is greater than 1.5× the characteristic diameter of a respective region. In still another variation, the center-to-center distance between regions is greater than 2× the characteristic diameter of a respective region. However, the center-to-center distance between regions can have another suitable value.

Furthermore, in variations, nanoparticle regions 130 and/or individual nanoparticles can be separated by material (e.g., a dielectric), in order to reduce critical distance requirements between nanoparticles and/or regions of nanoparticles. In examples, the dielectric material can include one or more of: glass, hafnium, silicon dioxide, aluminum oxide, and/or another suitable dielectric.

The regions 130 of nanoparticles can further be configured with a morphology or footprint that facilitates detection by an optical detection subsystem/imaging subsystem. In one variation, regions can be configured with a hexagonal footprint; however, in other variations, the regions can be configured with another suitable morphology (e.g., rectangular, circular, ellipsoidal, polygonal, amorphous, etc.)

### 3.2.1 Nanoparticle Functionalization

In variations, nanoparticle regions can be functionalized with one or more probe or affinity molecules for capturing target or non-target material from a sample.

Functionalization can be provided by antibodies (e.g., monoclonal, polyclonal, single-domain, etc.), single-chain variable fragments (scFvs), aptamers (e.g., DNA, RNA, XNA), nucleic acid primers, antigens, and/or other suitable functional components.

In one such variation, functionalization can apply high-selectivity antibody probes against SARS-CoV-2, SARS-CoV, MERS-CoV, other coronaviruses, influenza viruses, respiratory syncytial viruses, other antigens associated with respiratory diseases, other antigens associated with infectious diseases, and/or other antigens. In particular, functionalization with antibody probes (e.g., in comparison to aptamer probes) can be configured to provide high selectivity for different portions of coronaviruses (e.g., spike portions, nucleocapsid portions, etc.).

In another such variation, functionalization can be provided with antibodies (e.g., monoclonal antibodies, polyclonal antibodies, single-domain antibodies, etc.). In non-limiting examples, antibodies are specific for a cytokine or chemokine (e.g., one or more of interleukin-2 (IL-2); interleukin-4 (IL-4); interleukin-6 (IL-6); interleukin-10 (IL-10); interleukin-(IL-8); interleukin-12 (IL-12) interferon-gamma (IFN-γ); or tumor-necrosis-factor alpha (TNF-α)). Additional cytokines include, but are not limited to, acylation stimulating protein, adipokine, albinterferon, CCL1, CCL3, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL5, CCL6, CCL7, CCL8, CCL9, colony-stimulating factor, CX3CL1, CX3CR1, CXCL1, CXCL10, CXCL11, CXCL13, CXCL14, CXCL15, CXCL16, CXCL17, CXCL2, CXCL3, CXCL5, CXCL6, CXCL7, CXCL9, erythropoietin, Gc-MAF, granulocyte colony-stimulating factor, granulocyte macrophage colony-stimulating factor, hepatocyte growth factor, IL-17, IL1A, IL1B, inflammasome, interferome, interferon, interferon beta 1a, interferon beta 1b, interferon gamma, interferon type I, interferon type II, interferon type III, interferon-stimulated gene, interleukin 1 family (e.g., IL-1a, IL-1b), interleukin 1 receptor antagonist, interleukin 12, interleukin 12 subunit beta, interleukin 13, interleukin 15, interleukin 16, interleukin 2, interleukin 23, interleukin 23 subunit alpha, interleukin 34, interleukin 35, interleukin 7, interleukin 8, interleukin-36, leukemia inhibitory factor, leukocyte-promoting factor, lymphokine, lymphotoxin, lymphotoxin alpha, lymphotoxin beta, macrophage colony-stimulating factor, macrophage inflammatory protein, macrophage-activating factor, monokine, myokine, myonectin, nicotinamide phosphoribosyltransferase, oncostatin M, oprelvekin, platelet factor 4, proinflammatory cytokine, promegapoietin, RANKL, stromal cell-derived factor 1, talimogene laherparepvec, MMP8, granzyme B, HSP70, TNF-alpha, Ang-1, Ang-2, XCL1, XCL2, XCR1, and/or other suitable analytes.

Functionalization can be provided with respect to other indications detectable by way of an immunoassay approach, including one or more of: hemoglobin indications (e.g., for A1c level detection); hormonal indications (e.g., for detection and/or determining hormone concentrations associated with growth and development, sexual health and reproduction, mood, organ function, metabolism, etc.); and/or other indications (e.g., associated with testing whereby a user provides a sample from a remote location).

Functionalization can be provided with respect to different clinical panels (e.g., sexual health panels, cytokine release syndrome CAR-T, intensive care unit mortality with respect to sepsis/multiple organ dysfunction syndrome, asthma endotypes, cardiac failure, renal failure, preclinical biologic pharmacokinetics/pharmacodynamics, traumatic brain injury, acute kidney injury, other trauma, etc.).

In some embodiments, linkers are utilized to attach functional compounds to nanoparticle surfaces (e.g., using carbodiimide (e.g., EDC (1-Ethyl-3-(3-dimethylaminopropyl)-carbodiimide))/NHS chemistry). In some embodiments, the linker is a bifunctional thiol linker.

However, the sensor 110 can include other functional compounds and/or other linkers. Fabrication methods and linking of functional compounds are further described in Section 4 below.

### 3.2.2 Nanoparticle Regions for Various Applications

In variations, different nanoparticle regions can be optionally functionalized with different probe molecules 150 in order to provide additional functionality with respect to the assay(s) being performed using the sensor 100. For instance, in some embodiments, multiplex detection or other functionality is enabled by providing distinct probe/binding compounds in addressable locations (e.g., by physically addressed barcodes) associated with different nanoparticle regions. Additionally or alternatively, nanoparticles that are functionalized can be distributed across multiple regions (e.g., to provide controls, to provide other suitable functionality).

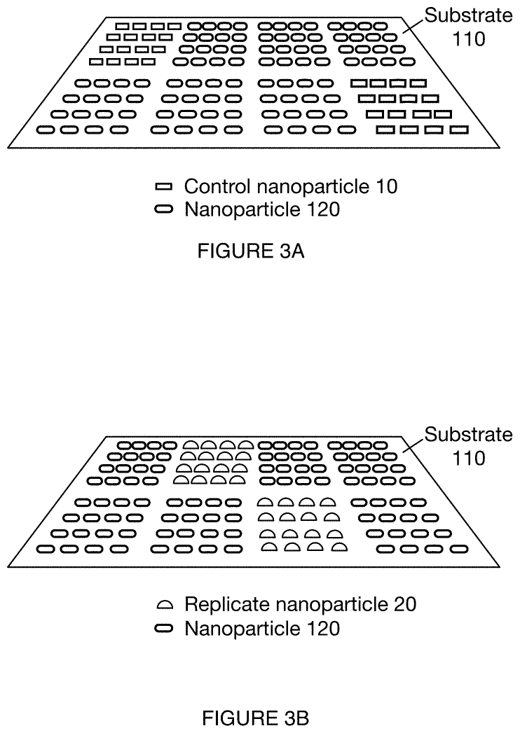

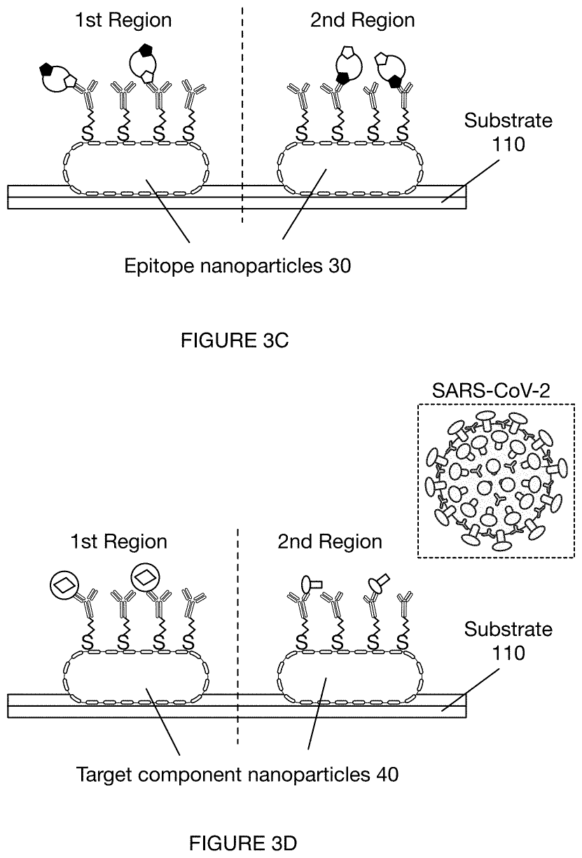

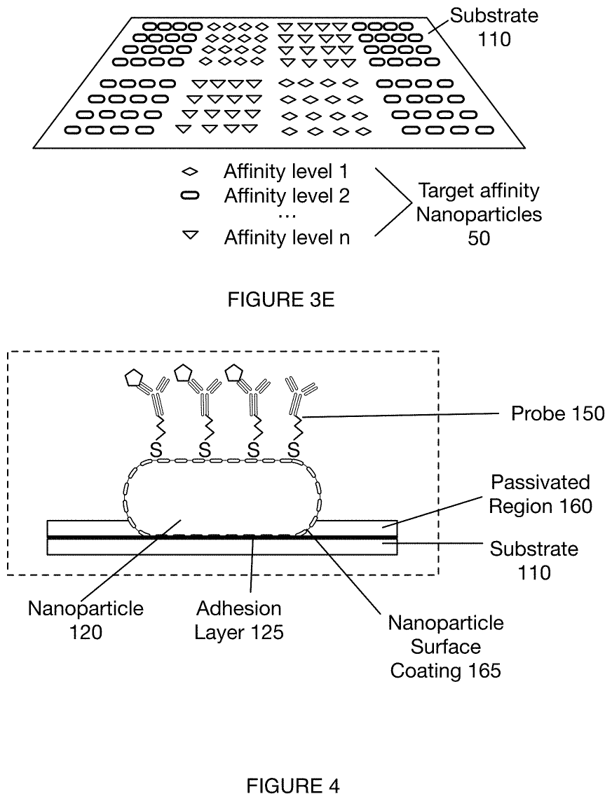

Controls: In one embodiment, as shown in FIG. 3A, the sensor 100 can include one or more (e.g. a subset of) control nanoparticles 10 functionalized with an inert probe (e.g., a probe that does not bind to anything in the target sample matrix), where the control nanoparticles 10 are used to calibrate the intensity of light as a function of position within the distribution of nanoparticles 120 of the sensor 110 (e.g., the control nanoparticles are positioned for calibration of intensity of light incident upon the sensor). The control nanoparticles 10 can also be used to determine the threshold for a positive detection (e.g. positive detection can be a function of the average of signals from control nanoparticles and a number of standard deviations from the average). As such, the control nanoparticles can be used to reduce systematic error due to inconsistent illumination within and between light sources. In variations, the control nanoparticles 10 can be distributed across all regions, in order to provide control signals from multiple or all portions of the sensor 100. In an example, the control nanoparticles 10 are distributed with 160 um spacing between control nanoparticles; however, in other examples, the control nanoparticles 10 can be otherwise spaced (e.g., uniformly spaced by another distance, non-uniformly spaced). In alternative variations, the control nanoparticles can, however, be clustered with respect to one or more regions of the sensor 100.

Replicates: Additionally or alternatively, in some embodiments, as shown in FIG. 3B, the sensor 100 can include one or more replicate regions 20 (e.g., regions including replicate nanoparticles) that can be used to determine an average signal for each type of probe molecule associated with a multiplexed assay. The replicate region(s) 20 can thus increase signal accuracy and reduce the impact of a single inaccurate or damaged sensor region. The replicate regions 20 can be distributed across the substrate no, in order to account for difference in illumination and/or other aspects (e.g., with respect to control nanoparticles 10); however, the replicate regions 20 can alternatively be grouped or clustered in a desired manner.

Epitopes: Additionally or alternatively, in some embodiments, as shown in FIG. 3C, the sensor 100 can include one or more epitope regions 30 (e.g., regions including epitope nanoparticles), such that multiple antibodies (e.g., different monoclonal antibodies, polyclonal antibodies) are used for detection of different spatial regions of a target, thereby increasing accuracy of detection. The epitope regions 30 can be distributed across the substrate 110, in order to account for difference in illumination and/or other aspects (e.g., with respect to control nanoparticles 10); however, the epitope regions 30 can alternatively be grouped or clustered in a desired manner.

Multiple targets: Additionally or alternatively, in some embodiments, as shown in FIG. 3D, the sensor 100 can include one or more target component regions 40, where the target component regions 40 are configured with different antibodies for different portions of target material of the sample. For instance, multiple antibodies against multiple target portions can be used to improve selectivity and, in the case of organism-derived targets, reduce the risk of a mutation or other change in the organism that reduces antibody affinity. With respect to the example of COVID-19 testing and targeting SARS-CoV-2 detection in samples, the target component regions 40 can include different antibodies for the spike portion of the virus, the nucleocapsid portion of the virus, receptor binding domain protein portions associated with the virus, and/or other virus portions. In related examples, the target component regions 40 can include antibodies specific to different portions of another virus (e.g., flu virus, coronavirus, etc.) and/or another organism. The target component regions 40 can be distributed across the substrate 110, in order to account for difference in illumination and/or other aspects (e.g., with respect to control nanoparticles 10); however, the target component regions 40 can alternatively be grouped or clustered in a desired manner.

Multiple affinities for the same target: Additionally or alternatively, in some embodiments, as shown in FIG. 3E, the sensor 100 can include one or more target affinity regions 50, where the target affinity regions 50 are configured with antibodies having varying affinities for a single target. In particular, probes having varying levels of affinity and having sensitivity to different concentration ranges of a target based on the affinity can be used to increase limits of detection with respect to different concentrations of the target within a particular sample. Thus, by using a range of affinities, the limits of detection of the target analyte are substantially increased, and the target affinity regions 50 can be used to quantify a concentration/amount of a target in a sample. The target affinity regions 50 can be distributed across the substrate 110, in order to account for difference in illumination and/or other aspects (e.g., with respect to control nanoparticles 10); however, the target affinity regions 50 can alternatively be grouped or clustered in a desired manner.

The sensor 100 can include one or all types of nanoparticles/regions described above, in a desired configuration.

### 3.3 Sensor—Other Aspects

3.3.1 Passivated Surfaces and/or Non-Specific Binding

Additionally or alternatively, in variations, unoccupied regions of the substrate 110 and/or nanoparticle surfaces can optionally include features to prevent undesired non-specific binding. For instance, as shown in FIG. 1, unoccupied regions of the substrate no and/or nanoparticle surfaces can include passivated surfaces 160 to prevent non-specific binding, without significantly shifting the LSPR wavelength, and in order to significantly improve signal-to-noise ratio (SNR) provided by the sensor 100. In particular, non-specific binding to the substrate no or surfaces of the nanoparticles 120 can compromise the performance of the sensor 100. For example: non-specific binding of the target antibody or target analyte to the substrate 100 reduces the amount of target available for detection by LSPR, therefore reducing limits of detection. Furthermore, non-specific binding of the target antibody or target analyte to the nanoparticle induces a shift in the LSPR spectrum, leading to either a false positive detection or reduced signal from specific binding. As such, prevention of non-specific binding is desired.

In one variation, the sensor 100 can include blocked/passivated regions 160, as shown in FIG. 4, to prevent non-specific binding to the substrate no. In one variation associated with the adhesion layer(s) 125 described above, the adhesion layer 125 (e.g., aminosilane adhesion layer) can be passivated/blocked with an inert coating to prevent non-specific binding. Preferably, the inert coating adheres to the substrate no in a manner that does not interfere with functionality of the distribution of nanoparticles. As such, the inert coating is preferably configured to not adhere to nanoparticle surfaces, is implemented in a manner in which it is fully replaced by the probe(s) bound to the nanoparticles 120, or is implemented in a manner in which it is partially replaced by the probe(s) and is characterized by sufficiently low molecular weight so as to not significantly shift the LSPR signal (e.g., ideally induces less than 5 nm peak shift). In an example, the inert coating comprises an N-Hydroxysuccinimide (NHS)-ester, which reacts with amine groups of the adhesion layer 125 without reacting with other sensor 100 components; however, other inert coatings can be used depending upon composition of the substrate 110 and/or adhesion layer 125.

Furthermore, the inert coating used for the blocked/passivated regions 160 is preferably characterized by low affinity for molecules present in the sample matrix. In examples, low-affinity functional groups include: alkanes, fluorocarbons, and polyethers; however, other low-affinity functional groups can be implemented. In a specific example, the low-affinity functional groups implemented include poly-ethyleneglycol chains; however, other low-affinity functional groups can be implemented.

In other variations, prevention of non-specific binding to the substrate 110 can include removal of portions of the adhesion layer 125 and/or other high-affinity coatings after nanoparticle deposition, where removal targets portions of the adhesion layer 125 that are not directly bound to the distribution of nanoparticles 120. In examples, removal be performed using one or more of: plasma treatment (e.g., by way of ozone, by way of oxygen, by way of air), UV irradiation, or another suitable method. With respect to removal of the adhesion layer, the deposited nanoparticles serve as a mask, allowing the coating directly bound to the gold to remain, while other portions are removed by suitable processes.

Additionally or alternatively, in variations, non-specific binding to the nanoparticle can be prevented by coating nanoparticle surfaces 165 (e.g., with a coating configured to adhere to the nanoparticle and prevent non-specific binding). In particular, the coating is preferably configured to adhere to the nanoparticle surfaces, but not to molecules in the sample matrix, and is preferably configured to not induce a significant shift in the LSPR spectrum (e.g., by being characterized by low molecular weight, by omitting any regions of high charge density, etc.). In an example, the nanoparticle coating can include a thiol that binds to gold nanoparticles. Additionally, in the example, the nanoparticle coating comprises one or more of: alkane, fluorocarbon, and polyether chains that block protein binding. However, other suitable blocking groups can be implemented.

### 3.4 Sensor—Channel

In variations, the sensor 100 can further include one or more channels 140 in fluid communication with the distribution of nanoparticles 120.

In some embodiments, the sensor 100 can further include one or more channels, where the one or more channels are in fluid communication with the distribution of coupled to the substrate 110. The one or more channels 140 function to transport assay components (e.g., samples, assay reagents, etc.) to the distribution of functionalized nanoparticles 120. In one embodiment, a single channel 140 can be implemented. However, in other variations, a set of channels can be implemented for transporting material to the distribution of functionalized nanoparticles 120. In some embodiments, the one or more channels have outlet and inlet components and/or reservoir components for supplying fluids to regions the sensor 100.

The one or more channels 140 can be provided in a manifold or other flow delivery device constructed of any suitable material. In examples, materials used can include one or more of: curable polyorganosiloxanes, and polyorganosiloxanes bearing methyl groups (e.g., polydimethylsiloxanes (“PDMS”)), other polymers (e.g., epoxy resins, curable polyurethane elastomers, etc.), polymer solutions (e.g., solutions of acrylate polymers in methylene chloride or other solvents), and/or other suitable polymeric or non-polymeric materials,

In one embodiment, the manifold or other flow delivery device can be configured as opaque device that scatters as little light as possible (e.g., as suitable for total internal reflectance), and in a specific example, the manifold or other flow delivery device includes a pressure sensitive adhesive with a graphite filler to provide opaqueness. In alternative embodiments, the manifold or other flow delivery device can be configured as a transparent device, in order to facilitate detection of signals emitted from the distribution of nanoparticles 120 upon reaction with sample material components. Additionally or alternatively, the manifold or other flow delivery device can be configured with suitable thermal properties to facilitate transport of materials to the distribution of nanoparticles 120 at a desired temperature with respect to an intended reaction. Additionally or alternatively, the manifold or other flow delivery device can be configured with suitable surface properties to prevent reactions and/or absorption of components of materials along the flow path to the distribution of nanoparticles 120.

Embodiments, variations, and examples of elements for supplying or driving fluid flow through the one or more channels 140 are further described in U.S. patent application Ser. No. 15/551,164 filed 16 Feb. 2016, which is incorporated by reference above.

### 4. Fabrication of Sensor

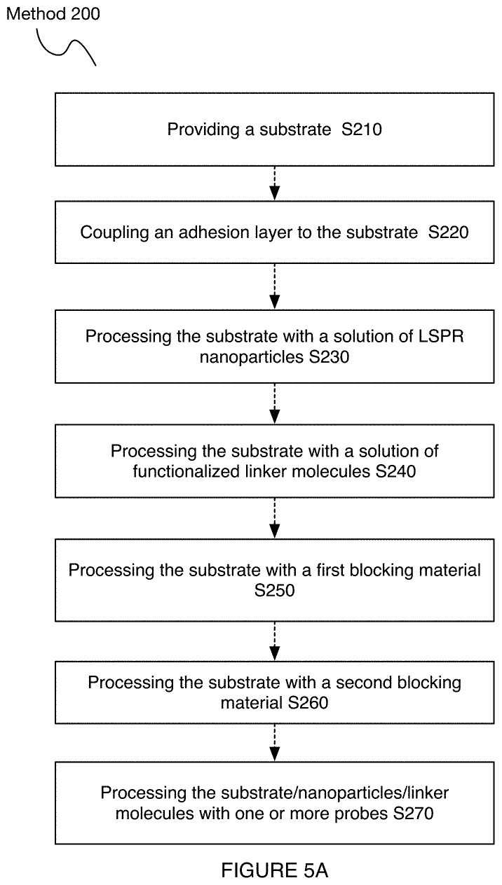

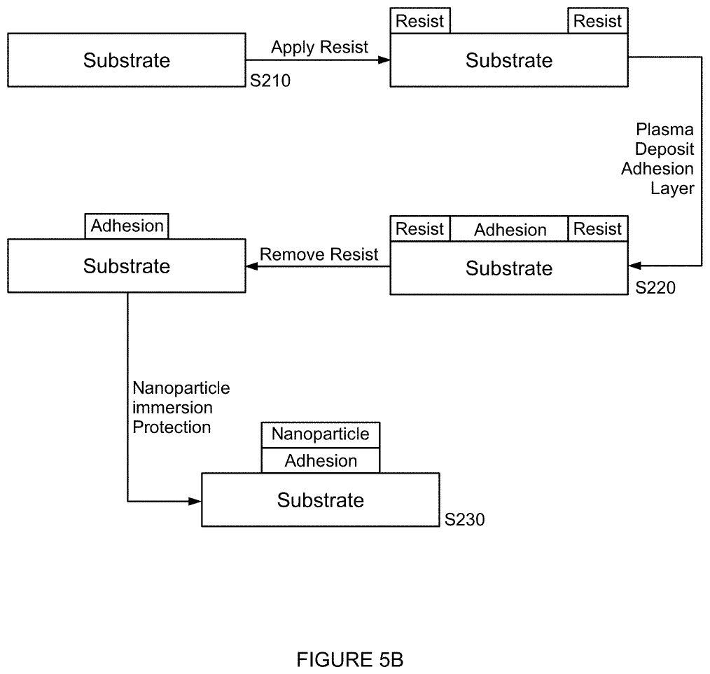

In one embodiment, as shown in FIGS. 5A and 5B, a method 200 for manufacturing a sensor includes: providing a substrate S210; coupling an adhesion layer to the substrate S220, wherein coupling the adhesion layer comprises patterning the adhesion layer onto the substrate; processing the substrate with a solution of nanoparticles characterized by localized surface plasmonic resonance (LSPR) behavior upon exposure to incident light, thereby adhering the nanoparticles to the adhesion layer in a pattern defining a set of regions S230; processing the substrate with a solution of functionalized linker molecules, thereby coupling the functionalized linker molecules to the nanoparticles at the substrate S240; processing the substrate with a first blocking material, thereby coupling the first blocking material to surfaces of the substrate S250 for prevention of non-specific binding to the substrate; processing the nanoparticles with a second blocking material, thereby coupling the second blocking material to surfaces of the nanoparticles S260 for prevention of non-specific binding to the nanoparticles; and processing the substrate with one or more solutions of probes, thereby providing functionality to different regions nanoparticles at the substrate for performance of one or more assays S270.

The method 200 functions to generate sensor units, each sensor including functionalized nanoparticle structures for LSPR, where binding of target material to the functionalized nanoparticle structures results in changes in local indices of diffraction. The changes in local indices of diffraction can then be detected, for performance of various assays. In variations, the method 200 can be used to manufacture a sensor for performing immunoassays and/or other types of assays involving detection of target material from a sample. In examples, the sensor can be manufactured with components configured to process biological samples (e.g., saliva samples, blood samples, urine samples, serum samples, other biological material) for detection of antibodies associated with respiratory diseases, such as antibodies against SARS-CoV-2.

In other variations, the sensor can be manufactured with components for applications including one or more of: therapeutic drug monitoring, infectious organism detection, diagnostics, detection of cancer biomarkers, detection of nutritional biomarkers, detection of genetic markers, detection of gene expression markers, detection of microRNA markers, hormone monitoring, fertility monitoring, serology testing for detection of various target antibodies (e.g., IgG antibodies, IgM antibodies, IgA antibodies, IgD antibodies, IgE antibodies), detection of cardiovascular health biomarkers, detection of gastrointestinal health biomarkers, detection of sexual health markers, detection of autoantibodies, detection of renal health biomarkers, and/or detection of other material associated with other conditions.

As shown in FIGS. 5A and 5B, fabrication of the sensor includes providing a substrate 110, which functions to provide a support for other components of the sensor and to provide a substrate that facilitates detection of optical signals by having suitable optical characteristics for transmission of light signals generated from the sensor. In one example, the substrate can be composed of a borosilicate glass (e.g., having a thickness of approximately 1.1 mm with a wafer diameter of greater than or equal to 200 mm); however, the substrate can alternatively be composed of material(s) and/or configured as described in Section 3.1 above.

As shown in FIGS. 5A and 5B, fabrication of the sensor also includes coupling an adhesion layer to the substrate S220, where the adhesion layer is applied to the substrate in a pattern defining one or more regions for nanoparticle coupling. The adhesion layer is preferably characterized by high affinity for the nanoparticles and in variations, can contain a functional group (e.g., amine, thiol, disulfide-modified, hydroxyl, carboxyl methyl, etc.); however, affinity for the nanoparticles can be achieved in another manner. In one example, the adhesion layer can be composed of (3-Aminopropyl)triethoxysilane (APTES) or another aminosilane; however, the adhesion layer can be composed of material(s) and/or configured as described in Section 3.2 above.

Coupling the adhesion layer to the substrate in Step S220 can include pattern-coating the adhesion layer to the substrate provided in Step S210. In one such variation of Step S220, photolithography can be used to apply a patterned resist onto the substrate, followed by deposition of the adhesion layer material (e.g., an aminosilane material) in regions exposed by the patterned resist. Step S220 can then include removal of the patterned resist, thereby leaving the adhesion layer coupled to the substrate in a desired pattern. However, patterning can be achieved using other suitable methods, including one or more of: another lithographical method (e.g., with positive patterning, with negative patterning); coating followed by etching (e.g., reactive ion etching) or removal of adhesion layer material to define a desired pattern; printing; and/or any other suitable patterning methods.

As shown in FIGS. 5A and 5B, fabrication of the sensor also includes processing the substrate with a solution of nanoparticles characterized by localized surface plasmonic resonance (LSPR) behavior upon exposure to incident light, thereby adhering the nanoparticles to the adhesion layer S230. In an example, the nanoparticles comprise gold nanorods (e.g. colloidal gold nanorods); however, in other variations, the nanoparticles can be composed of material(s) and/or configured as described in Section 3.2 above.

In Step S230, the solution of nanoparticles can include a solution of nanoparticles in glycerol and/or water, and processing in Step S230 can include immersion of the substrate with the patterned adhesion layer into the solution of nanoparticles, with agitation (e.g., rocking), drying, and incubation for a desired period of time. Outputs of Step S230 include an assembly in which the nanoparticles only adhere to the adhesion layer patterned onto the substrate.

As shown in FIGS. 5A and 5B, fabrication of the sensor also includes processing the substrate with a solution of functionalized linker molecules, thereby coupling the functionalized linker molecules to the nanoparticles at the substrate S240. The functionalized linker molecule functions to facilitate coupling of probes to the nanoparticle surfaces for selective binding to sample components, thereby enabling performance of various assays (e.g., single assays, multiplexed assays, etc.). In some embodiments, the functionalized linker molecules are utilized to attach functional compounds to nanoparticle surfaces (e.g., using carbodiimide (e.g., EDC (1-Ethyl-3-(3-dimethylaminopropyl)-carbodiimide))/NHS chemistry). In variations, the functionalized linker molecules comprise bifunctional thiol linker, and in a specific example, the functional linker molecules include molecules having a long alkane chain and carboxyl containing thiols (e.g., 11-mercaptoundecanoic acid, etc.). However, in other variations, the functional linker molecules can be composed of material(s) and/or configured as described in Section 3.2 above.

In Step S240, the solution of functional linker molecules can include a solution of functional linker molecules provided in ethanol, and processing in Step S240 can include immersion of the substrate with the patterned adhesion layer and coupled nanoparticles into the solution of functional linker molecules, with agitation (e.g., rocking) and drying. Outputs of Step S240 include an assembly in which the functional linker molecules are coupled to the nanoparticles at the substrate.

As shown in FIGS. 5A and 5B, fabrication of the sensor also includes processing the substrate with a first blocking material, thereby coupling the first blocking material to surfaces of the substrate S250 for prevention of non-specific binding to the substrate. The first blocking material functions to coat/passivate surfaces to prevent non-specific binding, without significantly shifting the LSPR wavelength, and in order to significantly improve signal-to-noise ratio (SNR) provided by the sensor.

Processing the substrate with the first blocking material in Step S250 can include applying an inert coating to exposed portions of the substrate in a manner that does not interfere with functionality of the distribution of nanoparticles. A such, I=in variations, the inert coating can be applied in a manner in which it does not adhere to nanoparticle surfaces, can be applied in a manner in which it is fully replaced by the probe(s) bound to the nanoparticles in Step S270, or is implemented in a manner in which it is partially replaced by the probe(s) of Step S270 and is characterized by sufficiently low molecular weight so as to not significantly shift the LSPR signal (e.g., ideally induces less than 5 nm peak shift). Furthermore, the inert coating can be characterized by low affinity for molecules present in the sample matrix. In examples, low-affinity functional groups include: alkanes, fluorocarbons, and polyethers; however, other low-affinity functional groups can be implemented. In a specific example, the low-affinity functional groups implemented include poly-ethyleneglycol chains; however, other low-affinity functional groups can be implemented. However, in other variations, the first blocking material can be composed of material(s) and/or configured as described in Section 3.3.1 above.

In an example, the inert coating comprises an N-Hydroxysuccinimide (NHS)-ester (e.g., sulfo-NHS), which reacts with groups (e.g., amine groups) of the adhesion layer without reacting with other sensor components; however, other inert coatings can be used depending upon composition of the substrate 110 and/or adhesion layer. In Step S250, processing with the first blocking material can include coating the substrate with the first blocking material, with agitation (e.g., rocking), flushing, and drying. Outputs of Step S250 include an assembly in which exposed portions of the substrate (e.g., non-nanoparticle-coupled surfaces) are coated with the first blocking material.

While implementation of a blocking material can be used in Step S250, in other variations, prevention of non-specific binding to the substrate can additionally or alternatively include removal of portions of the adhesion layer and/or other high-affinity coatings after nanoparticle deposition, where removal targets portions of the adhesion layer that are not directly bound to the distribution of nanoparticles 120. In examples, removal be performed using one or more of: plasma treatment (e.g., by way of ozone, by way of oxygen, by way of air), UV irradiation, or another suitable method. With respect to removal of the adhesion layer, the deposited nanoparticles serve as a mask, allowing the coating directly bound to the gold to remain, while other portions are removed by suitable processes.

As shown in FIGS. 5A and 5B, fabrication of the sensor can also include processing the nanoparticles with a second blocking material, thereby coupling the second blocking material to surfaces of the nanoparticles S260 for prevention of non-specific binding to the nanoparticles. The second blocking material functions to block nanoparticle surfaces to prevent non-specific binding, without inducing a significant shift in the LSPR spectrum (e.g., by being characterized by low molecular weight, by omitting any regions of high charge density, etc.).

In variations, the second blocking material can include a thiol that binds to gold nanoparticles. Additionally, in the example, the nanoparticle coating comprises one or more of: alkane, fluorocarbon, and polyether chains that block protein binding. However, in other variations, the second blocking material can be composed of material(s) and/or configured as described in Section 3.3.1 above.

In Step S260, processing with the second blocking material can include coating the nanoparticle surfaces of the sensor with the second blocking material, with agitation (e.g., rocking), flushing, and drying. Outputs of Step S260 include an assembly in which nanoparticle surfaces are coated with the second blocking material.

As shown in FIGS. 5A and 5B, fabrication of the sensor also includes processing the substrate with one or more solutions of probes, thereby providing functionality to different regions nanoparticles at the substrate for performance of one or more assays S270. Step S270 functions to provide nanoparticle regions arranged for performance of multiplexed assays, with one or more of: control regions, replicate regions, epitope regions, regions configured for multiple targets, regions configured with components having different affinities for the same target, and/or other suitable regions. Probe/functional molecules for binding and detection of target material from a sample are described in Section 3.2.1 above.

In Step S270, processing with the one or more solutions of probes can include applying probe solutions (e.g., antibody solutions, antigen solutions, aptamer solutions, etc.) to the nanoparticles with functional linker molecules, where the solutions are provided with suitable buffers having suitable pH characteristics, with suitable incubation times, suitable agitation times, and suitable drying times. Processing can be performed in sequence, in order to couple different types of functional probes to different nanoparticle regions in Step S270, where different types of probes and corresponding functionalities are described in Section 3.2.2 above.

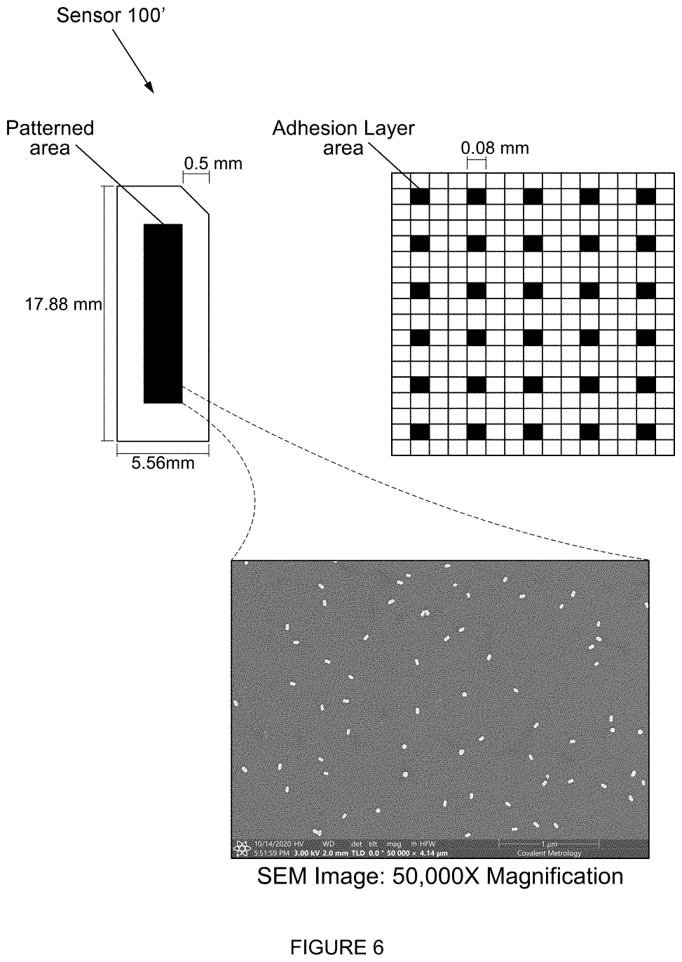

FIG. 6 depicts a specific example of a portion of a sensor 100′ produced by an embodiment of the method 200 described above.

While steps are described in an order above, in variations, steps of the method 200 can alternatively be performed in another suitable manner. Furthermore, units of the sensor can be fabricated in batches, followed by scoring to separate individual units; however, fabrication can alternatively be performed in another suitable manner.

### 5. Conclusions

The FIGURES illustrate the architecture, functionality and operation of possible implementations of systems and/or methods a according to preferred embodiments, example configurations, and variations thereof. In this regard, each block in the flowchart or block diagrams may represent a module, segment, or portion of a process or code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the preferred embodiments of the invention without departing from the scope of this invention defined in the following claims.

What is claimed is:

1. A sensor comprising:
a transparent substrate;
an adhesion layer patterned onto the transparent substrate; and
a distribution of nanoparticles coupled to the adhesion layer and configured as a set of regions, wherein the distribution of nanoparticles is functionalized with a set of probes specific for target material of a sample, and wherein the set of probes is distributed across the set of regions.

2. The sensor of claim 1, wherein the transparent substrate is structured for transmission of light within a wavelength range corresponding to wavelengths in which the distribution of nanoparticles absorb light.

3. The sensor of claim 1, wherein the transparent substrate is configured with a refractive index greater than 1.3.

4. The sensor of claim 1, wherein the adhesion layer is composed of an aminosilane material.

5. The sensor of claim 1, wherein the distribution of nanoparticles is arranged as a monodisperse distribution of nanoparticles, with spacing between adjacent nanoparticles by a critical distance configured to prevent electromagnetic coupling between adjacent nanoparticles of the distribution.

6. The sensor of claim 1, wherein the distribution of nanoparticles has a density greater than 10 nanoparticles/μm², and a level of aggregation in which less than 10% of nanoparticles are positioned within 250 nm of another nanoparticle of the distribution.

7. The sensor of claim 1, wherein the distribution of nanoparticles comprises a subset of control nanoparticles functionalized with inert probes, the subset of control nanoparticles distributed across the set of regions and positioned for calibration of intensity of light incident upon the sensor.

8. The sensor of claim 1, wherein the distribution of nanoparticles comprises a subset of replicate nanoparticles configured for determination of an average signal for each probe type of the set of probes.

9. The sensor of claim 1, wherein the distribution of nanoparticles comprises a subset of epitope nanoparticles functionalized with multiple antibody epitopes configured for detection of different spatial regions of said target material of the sample.

10. The sensor of claim 1, wherein the set of regions comprises one or more target component regions comprising nanoparticles functionalized with a set of antibodies with affinities for different portions of said target material of the sample.

11. The sensor of claim 10, wherein the set of antibodies comprises a first subset of antibodies with affinity for a spike portion of SARS-CoV-2, a second subset of antibodies with affinity for a nucleocapsid portion SARS-CoV-2, and a third subset of antibodies with affinity for receptor binding domain protein portions of SARS-CoV-2.

12. The sensor of claim 1, wherein the set of regions comprises one or more target affinity regions comprising nanoparticles functionalized with a set of antibodies having varying affinities for a single target of the sample.

13. The sensor of claim 1, wherein exposed regions of the substrate are passivated with an inert coating having low affinity for target material of the sample and configured for prevention of non-specific binding.

14. The sensor of claim 1, wherein surfaces of the distribution of nanoparticles are treated with a coating configured for prevention of non-specific binding to the distribution of nanoparticles.

15. The sensor of claim 14, wherein the coating comprises a thiol with affinity for binding to gold nanoparticles.

16. A sensor comprising:
a transparent substrate;
an adhesion layer composed of an aminosilane patterned onto the transparent substrate; and
a distribution of gold nanoparticles coupled to the adhesion layer and configured as a set of regions, and wherein the set of regions comprises one or more target component regions comprising gold nanoparticles functionalized with a set of antibodies with affinities for different portions of a target material of the sample,
wherein exposed regions of the substrate are passivated with an inert coating having low affinity for the target material of the sample and configured for prevention of non-specific binding.

17. The sensor of claim 16, wherein the transparent substrate is structured for transmission of light within a wavelength range corresponding to wavelengths in which the distribution of gold nanoparticles absorb light, and wherein the transparent substrate is configured with a refractive index greater than 1.

18. The sensor of claim 16, wherein the distribution of gold nanoparticles is arranged as a monodisperse distribution of gold nanoparticles, with spacing between adjacent nanoparticles by a critical distance configured to prevent electromagnetic coupling between adjacent gold nanoparticles of the distribution.

19. The sensor of claim 16, wherein the set of antibodies comprises a first subset of antibodies with affinity for a spike portion of SARS-CoV-2, a second subset of antibodies with affinity for a nucleocapsid portion SARS-CoV-2, and a third subset of antibodies with affinity for receptor binding domain protein portions of SARS-CoV-2.

20. The sensor of claim 16, wherein the distribution of gold nanoparticles comprises a subset of control nanoparticles functionalized with inert probes, the subset of control nanoparticles distributed across the set of regions and positioned for calibration of intensity of light incident upon the sensor.
